(19) United States
(12) Patent Application Publication
(10) Pub. No.: US 2017/0173259 A1
(43) Pub. Date: Jun. 22, 2017

(54) System of Intravenous Fluid Temperature Control

(71) Applicant: Gregory D. Cullen, Sanford, NC (US)

(72) Inventor: Gregory D. Cullen, Sanford, NC (US)

(21) Appl. No.: 15/389,330

(22) Filed: Dec. 22, 2016

Related U.S. Application Data
(60) Provisional application No. 62/270,679, filed on Dec. 22, 2015.

(51) Int. Cl. A61M 5/142; A61M 25/06

(52) U.S. Cl. CPC A61M 5/14228; A61M 25/0606; A61M 2205/3368; A61M 2230/50; A61M 2205/502; A61M 2205/8262; A61M 2205/3653; A61M 2205/3334

(57) ABSTRACT

A system of intravenous fluid temperature control is a combination of apparatuses that provides a two-stage temperature control of intravenous (IV) fluid. The combination of apparatuses includes an IV fluid retention unit, a microcontroller, at least one heating mechanism, a temperature sensor, and a portable power source. The IV fluid retention unit contains the IV fluid which is delivered into a patient. The at least one heating mechanism and the temperature sensor is in thermal communication with the IV fluid retention unit. The microcontroller is electronically connected to the at least one heating mechanism and the temperature sensor. The microcontroller, the at least one heating mechanism, the temperature sensor, and the portable power source is externally mounted on the IV fluid retention unit. The portable power source is electrically connected to the microcontroller, the at least one heating mechanism, and the temperature sensor.

The current application claims priority to U.S. provisional application Ser. No. 62/270,679 filed on Dec. 22, 2015.

### FIELD OF THE INVENTION

The present invention generally relates to a system of intravenous fluid temperature control. More specifically, the present invention regulates the temperature of intravenous fluid while in storage, transport, and the point of entry into a patient's body.

### BACKGROUND OF THE INVENTION

Although a variety of intravenous temperature control devices exist today, none are like the present invention in that they do not provide a two-stage temperature control system. Additionally, most existing intravenous temperature control devices are only capable of heating the intravenous fluid, they do not provide a cooling feature. Therefore, the present invention provides a unique temperature control system that is unlike any of the existing intravenous temperature control systems that exist today.

The present invention is capable of storing and regulating the temperature of a plurality of intravenous fluid bags at the same time. The temperature control system of the present invention provides a much more reliable and consistent method of controlling the temperature of the intravenous fluid bags as both the carrying case and intravenous fluid storage packs are capable of monitoring and automatically adjusting the temperature within. In this regard, the two devices act as a fail-safe system as the present invention is capable of controlling the temperature even if one of the devices fails. Therefore, the present invention provides an improved intravenous fluid temperature control system when compared to similar existing inventions.

### BRIEF DESCRIPTION OF THE DRAWINGS

FIG. 1 is a perspective view of an embodiment of the present invention, wherein the bag is shown with the inseparable housing and the fluid transfer line.

FIG. 2 is a perspective view of an embodiment of the present invention, wherein the separable housing is detached from the bag, and the bag is in fluid communication with the fluid transfer line.

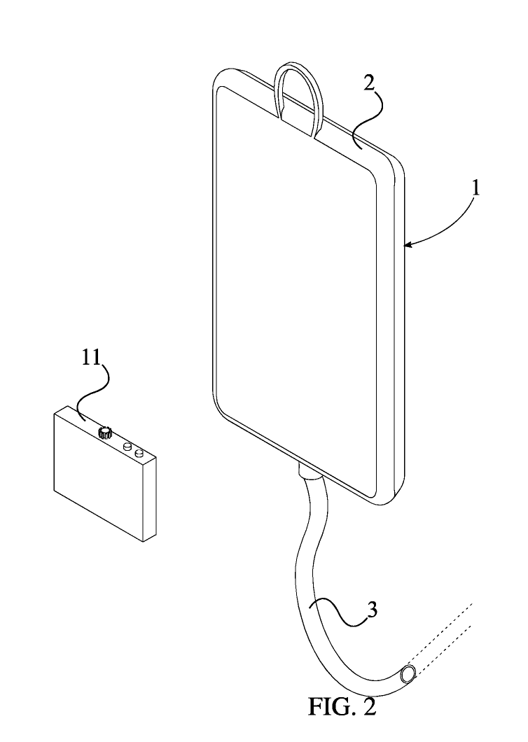

FIG. 3 is a schematic view of the electronic connection between the microcontroller, the at least one heating mechanism, and the temperature sensor of an embodiment of the present invention.

FIG. 4 is a schematic view of the electrical connection between the microcontroller, the at least one heating mechanism, the temperature sensor, and the portable power source of an embodiment of the present invention.

FIG. 5 is a schematic view of the engagement between the microcontroller, the at least one heating mechanism, the temperature sensor, and the portable power source about the IV retention unit of an embodiment of the present invention.

FIG. 6 is a schematic view of the electronic connection between the user input/output console and the microcontroller of an embodiment of the present invention.

FIG. 7 is a schematic view of the engagement between the microcontroller, the at least one heating mechanism, the temperature sensor, the portable power source, and the user input/output console about the IV retention unit of an embodiment of the present invention.

FIG. 8 is a schematic view of the engagement between the microcontroller, the at least one heating mechanism, the temperature sensor, and the portable power source within the inseparable housing with that of the bag of an embodiment of the present invention.

FIG. 9 is a schematic view of the engagement between the microcontroller, the at least one heating mechanism, the temperature sensor, and the portable power source within the separable housing with that of the bag of an embodiment of the present invention.

FIG. 10 is a schematic view of the insulative sheath, the plurality of heating wires, and the fluid transfer line of an embodiment of the present invention.

FIG. 11 is a schematic view of the engagement of the bag, the peristaltic pump, the fluid transfer line, and the flowrate sensor of an embodiment of the present invention.

FIG. 12 is a schematic view of the electronic connection between the microcontroller and the flowrate sensor of an embodiment of the present invention.

FIG. 13 is a perspective view of an embodiment of the present invention, wherein the bag is positioned within the sleeve, and the bag is exposed via the sealing mechanism.

FIG. 14 is a perspective view of an embodiment of the present invention, wherein the fluid transfer line exits the sleeve through the line-receiving hole.

FIG. 15 is a schematic view of the engagement between the microcontroller, the at least one heating mechanism, and the temperature sensor within the sleeve of an embodiment of the present invention.

FIG. 16 is a perspective view of an embodiment of the present invention, wherein the plurality of sleeves is electronically connected to the climate-controlled storage container.

FIG. 17 is a perspective view of an embodiment of the present invention, wherein a sleeve is electronically connected to the climate-controlled storage container, and the remaining plurality of power connectors is disengaged.

FIG. 18 is a schematic view of the wireless communication between the IV retention unit and the climate-controlled storage container of an embodiment of the present invention.

### DETAILED DESCRIPTION OF THE INVENTION

All illustrations of the drawings are for the purpose of describing selected versions of the present invention and are not intended to limit the scope of the present invention.

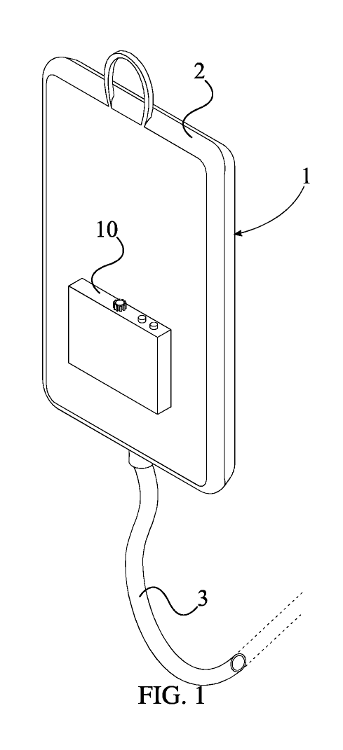

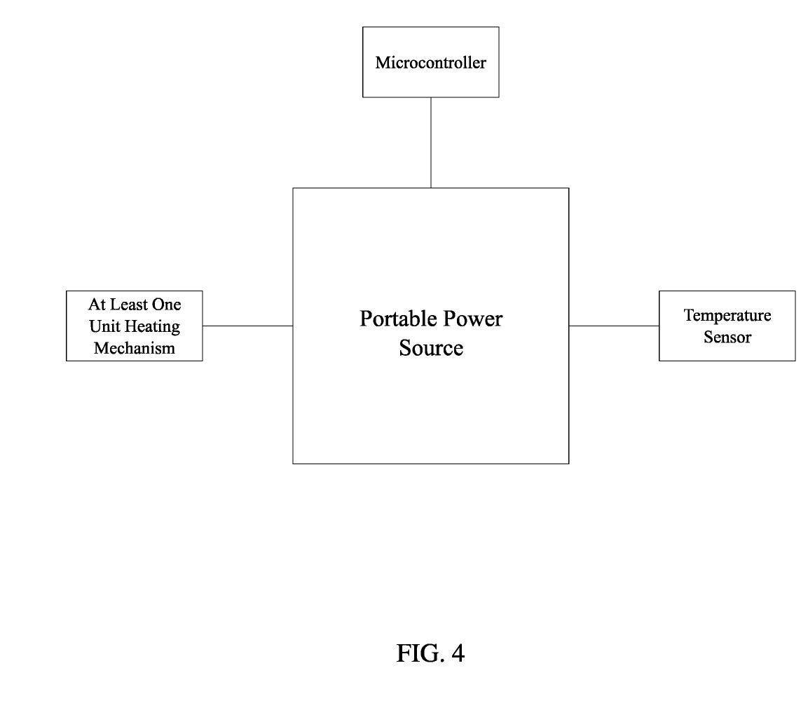

The present invention is a system of intravenous (IV) fluid temperature control that allows a user to regulate the temperature of IV fluid while in storage, transport, and at the point of entry into a patient's body. The present invention further provides two-stage temperature control of IV fluid before the IV fluid enters the patient's body. The present invention constantly notifies the temperature of the IV fluid to a user and allows the user to change the temperature if the temperature is too hot or too cold. Consequently, the present invention provides both heating and cooling mechanisms so that the IV fluid may be quickly and evenly heated. As seen in FIG. 1 and FIG. 4, the present invention comprises an IV fluid retention unit 1, a microcontroller 4, at least one heating mechanism 5, a temperature sensor 7, and a portable power source 8. The IV fluid retention unit 1 houses the IV fluid. The microcontroller 4 controls the at least one heating mechanism 5. More specifically, the microcontroller 4 outputs the temperatures for the at least one heating mechanism 5 as the microcontroller 4 calculates the necessary changes in temperature according to the temperature sensor 7. The temperature sensor 7 measures the current temperature of the IV fluid bag 2 and delivers the temperature reading to the microcontroller 4. The portable power source 8 not only provides power to the microcontroller 4, the at least one heating mechanism 5, and the temperature sensor 7 but aids in the temperature control of the IV fluid retention unit 1 due to the current delivered to microcontroller 4, the at least one heating mechanism 5, and the temperature sensor 7, respectively. The communication between the IV fluid retention unit 1, the microcontroller 4, the at least one heating mechanism 5, the temperature sensor 7, and the portable power source 8 provide a faster, more accurate, and thorough temperature control of IV fluid.

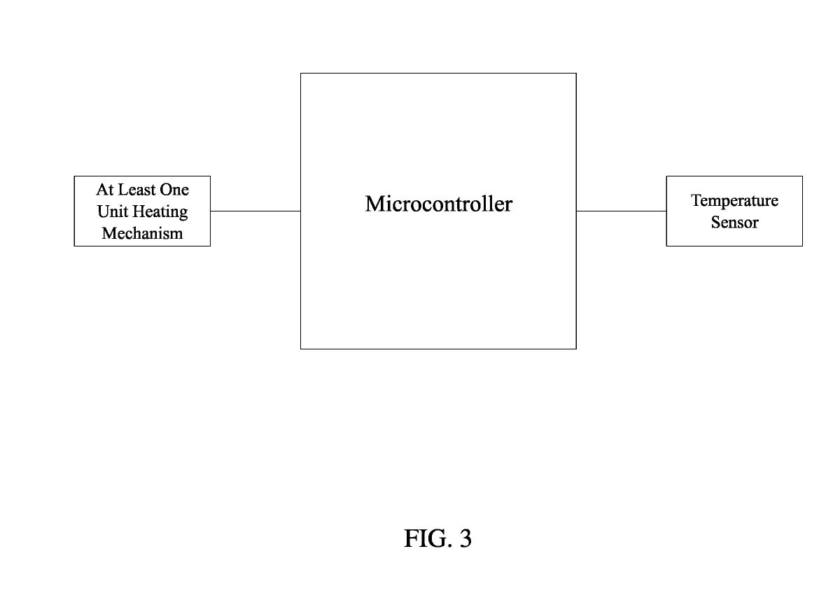

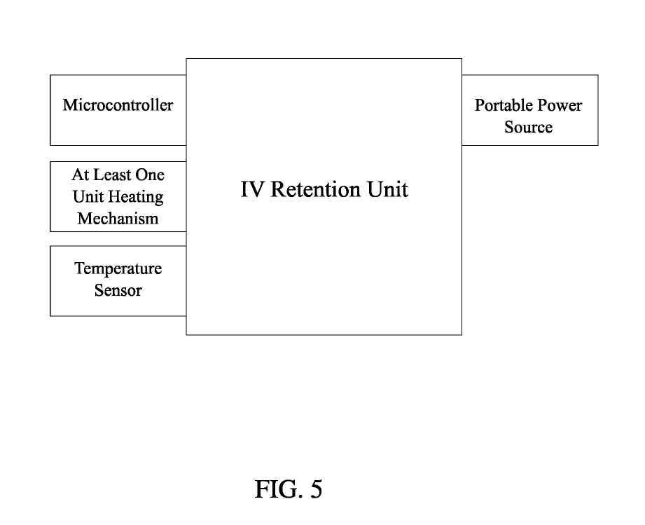

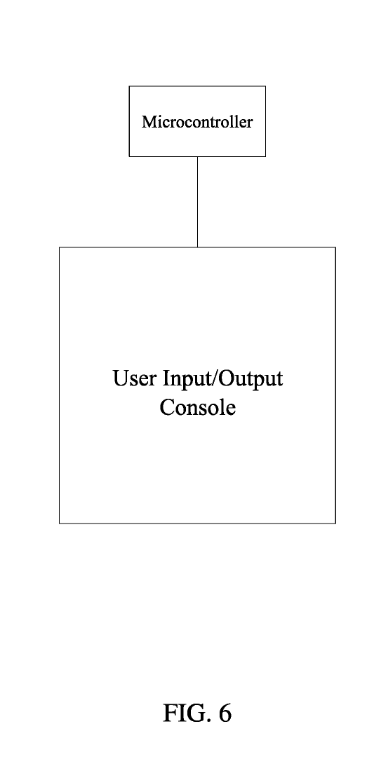

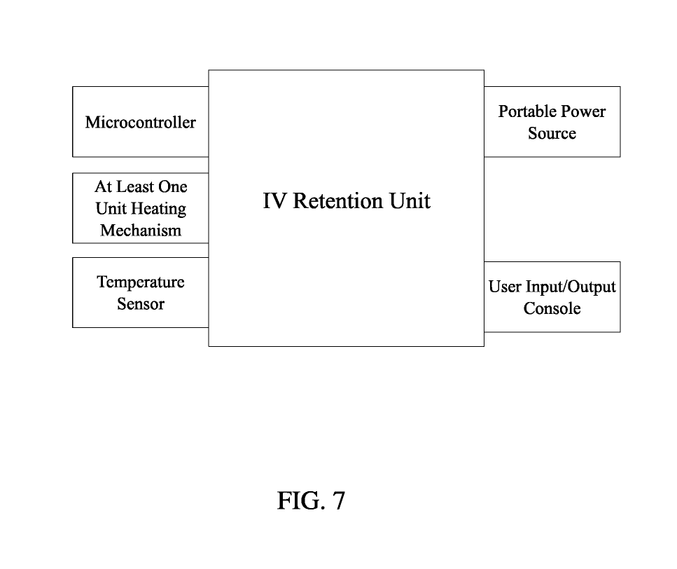

The overall configuration of the aforementioned components allows a user to adjust the current temperature of the IV fluid and maintain the desired temperature of the IV fluid. The at least one heating mechanism 5 and the temperature sensor 7 are in thermal communication with the IV fluid retention unit 1, as shown in FIG. 5. The heating mechanism 5 adjusts the temperature of the IV fluid by delivering heat to the IV retention unit 1 so that the IV fluid reaches the desired temperature. The temperature sensor 7 receives the heat of the IV fluid within the IV retention unit 1 and outputs the current temperature to the microcontroller 4 so that the microcontroller 4 may calculate the necessary changes in temperature of the heating mechanism 5. Consequently, the microcontroller 4 is electronically connected to the at least one heating mechanism 5 and the temperature sensor 7, as shown in FIG. 3, so that the at least one heating mechanism 5 receives the most accurate temperature adjustments according to the current temperature of the IV fluid via the temperature sensor 7. The portable power source 8 is electrically connected to the microcontroller 4, the at least one heating mechanism 5, and the temperature sensor 7, as shown in FIG. 4, so that each may function while in use with a patient, as shown in FIG. 4. The microcontroller 4, the at least one heating mechanism 5, the temperature sensor 7, and the portable power source 8 are all externally mounted to the IV fluid retention unit 1 so that each of these components does not get damaged by either the liquid of the IV fluid or the temperature of the IV fluid. In order for the user to control the temperature of the IV fluid, a user input/output console 9 is externally mounted onto the IV fluid retention unit 1 in the preferred embodiment of the present invention, as shown in FIG. 7. Furthermore, the user input/output console 9 is electronically connected to the microcontroller 4 so that the user may manually input the desired temperature of the IV fluid into the microcontroller 4 and so that the microcontroller 4 may calculate the necessary temperatures and temperature changes of the at least one heating mechanism 5. This electronic connection between the user input/output console 9 and the microcontroller 4 is shown in FIG. 6.

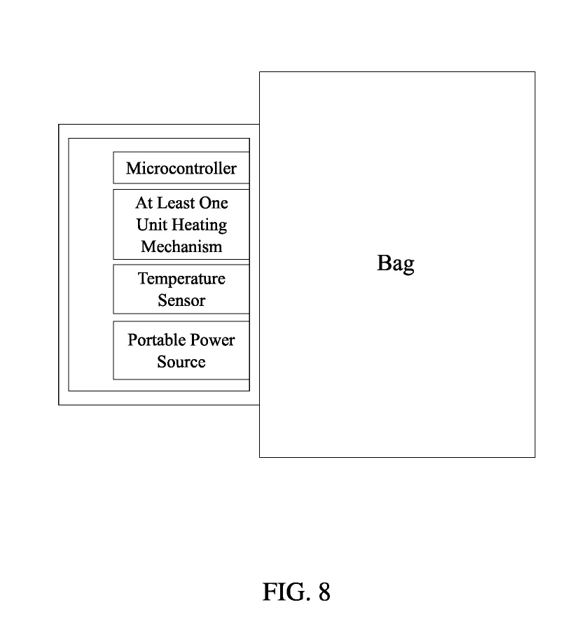

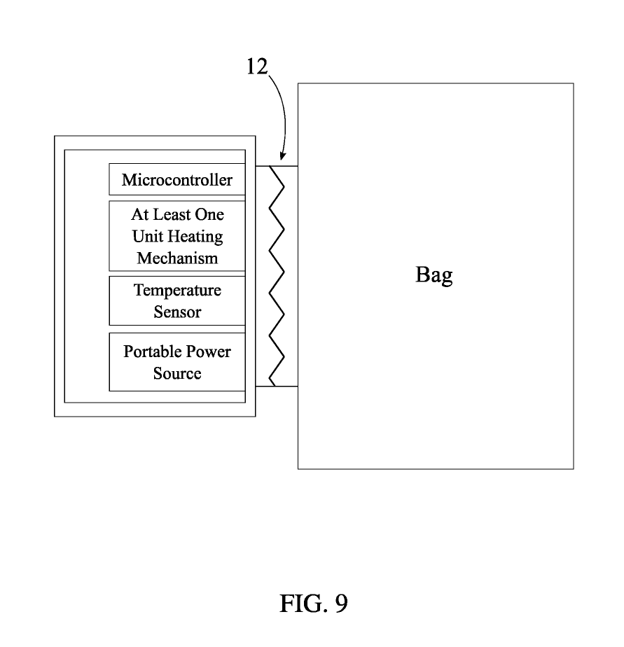

In an embodiment of the present invention, an inseparable housing 10 protects the microcontroller 4, the at least one heating mechanism 5, the temperature sensor 7, and the portable power source 8 while these components are mounted to the IV fluid retention unit 1. More specifically, the IV fluid retention unit 1 comprises a bag 2, which retains the IV fluid, and the inseparable housing 10 is externally connected onto the bag 2 so that the IV fluid does not come into contact with the inseparable housing 10, as shown in FIG. 8. The bag 2 not only houses the IV fluid, but prevents any contamination of the IV fluid. Furthermore, the microcontroller 4, the at least one heating mechanism 5, the temperature sensor 7, and the portable power source 8 are mounted within the inseparable housing 10. In another embodiment of the present invention, a separable housing 11 may also protect the microcontroller 4, the at least one heating mechanism 5, the temperature sensor 7, and the portable power source 8 while connected and disconnected from the bag 2. In this embodiment, the separable housing 11 is detachably attached to the bag 2 via a disengageable fastener 12. The disengageable fastener 12 is preferably a pair of hook-and-loop fastening patches fixed to the separable housing and the bag, respectively, as shown in FIG. 9. Similar to the inseparable housing 10, the microcontroller 4, the at least one heating mechanism 5, the temperature sensor 7, and the portable power source 8 is mounted within the separable housing 11. In this embodiment, the microcontroller 4, the at least one heating mechanism 5, the temperature sensor 7, and the portable power source 8 communicate with one another and the bag 2 so that the IV fluid within the bag 2 is effectively heated.

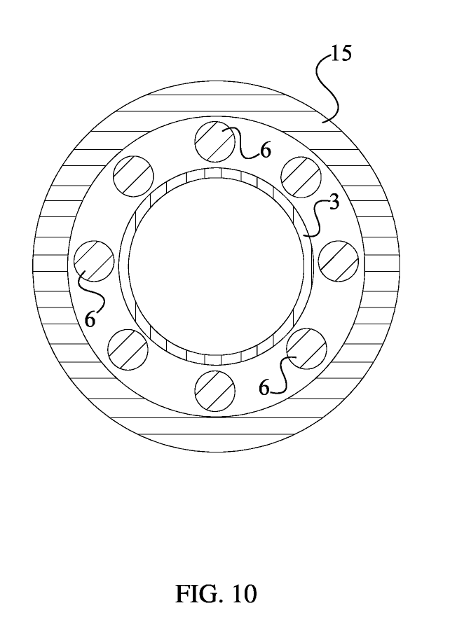

In order to provide more control of the temperature of the IV fluid as it enters a patient's body, the IV fluid retention unit 1 may further comprise a bag 2 and a fluid transfer line 3 in the preferred embodiment of the present invention. The fluid transfer line 3 delivers the IV fluid to the patient's body. In this embodiment, the at least one heating mechanism 5 comprises a plurality of heating wires 6, as shown in FIG. 10. The plurality of heating wires 6 maintains and adjusts the temperature of the IV fluid as the IV fluid traverses through the fluid transfer line 3. The arrangement between the bag 2 and the fluid transfer line 3 is such that fluid transfer line 3 is in fluid communication with the bag 2 so that the IV fluid within the bag 2 safely reaches and enters a patient's body. The arrangement between the plurality of heating wires 6 and the fluid transfer line 3 is such that the plurality of heating wires 6 is positioned along and distributed around the fluid transfer line 3, effectively heating the IV fluid up to the point of entry into the patient's body. More specifically, the plurality of heating wires 6 is laterally mounted to the fluid transfer line 3 so that the plurality of heating wires 6 is consistently warming the fluid transfer line 3.

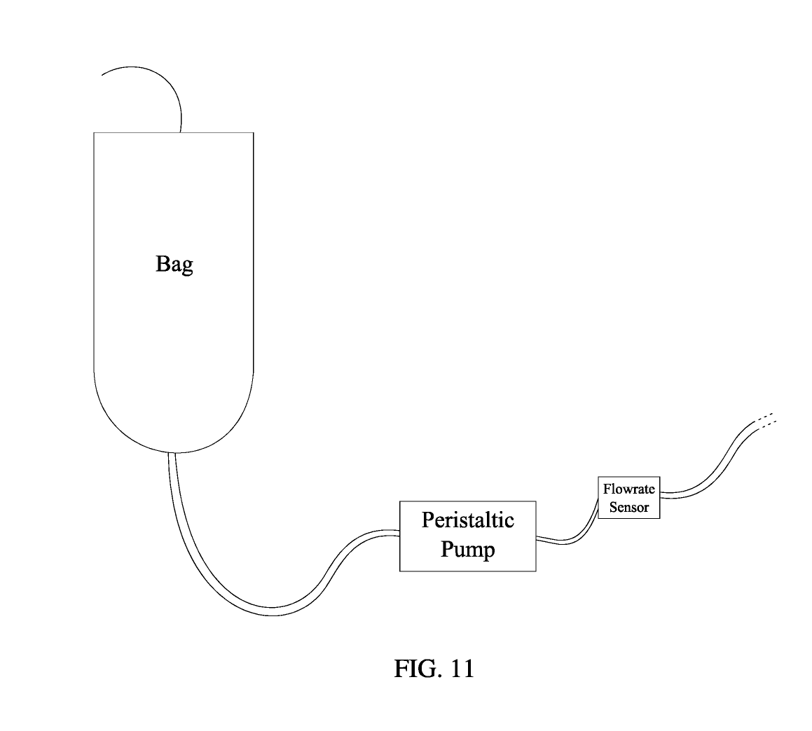

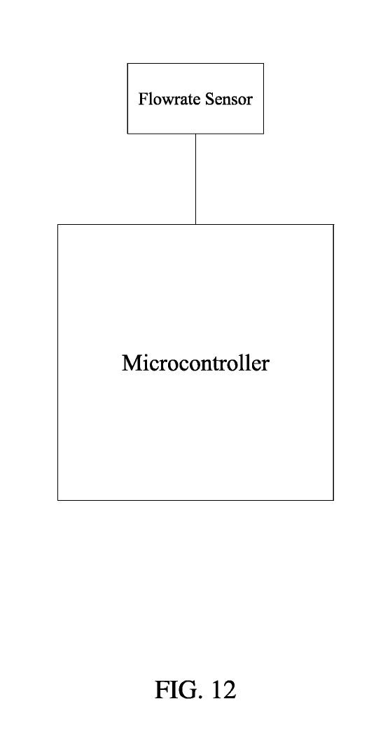

In an embodiment of the present invention, a peristaltic pump 13 controls the flowrate of the IV fluid as it traverses through the fluid transfer line 3. The microcontroller 4 is electronically connected to the peristaltic pump 13, as is the microcontroller 4 to the at least one heating mechanism 5 and temperature sensor 7 in the preferred embodiment of the present invention. Furthermore, the fluid transfer line 3 is in fluid communication with the bag 2 through the peristaltic pump 13 so that the flowrate of the IV fluid exiting the bag 2 is more accurately controlled before it enters the peristaltic pump 13, as shown in FIG. 11, thereby controlling the temperature of the IV fluid. In this embodiment of the present invention, not only is the current temperature of the IV fluid within the bag 2 determined by the microcontroller 4, but the flowrate of the IV fluid as it exits the bag 2 and into the fluid transfer line 3 is determined by the flowrate sensor 14. The microcontroller 4 is electronically connected to the flowrate sensor 14. This connection, as shown in FIG. 12, allows the flowrate sensor 14 to measure the current flowrate of the IV fluid and deliver the flowrate reading to the microcontroller 4 so that the microcontroller 4 may accurately calculate the necessary temperature changes of the at least one heating mechanism 5. More specifically, the flowrate sensor 14 is operatively integrated along the fluid transfer line 3, wherein the flowrate sensor 14 is used to measure a fluid flowrate through the fluid transfer line 3. In an alternate embodiment of the present invention, an insulative sheath 15 may further manage the temperature of the IV fluid as it traverses through the fluid transfer line 3. The fluid transfer line 3 and the plurality of heating wires 6 are enclosed within the insulative sheath 15, as shown in FIG. 10, thereby regulating the temperature of the IV fluid traversing through the fluid transfer line 3 and securing the plurality of heating wires 6 around the fluid transfer line 3. The insulative sheath 15 manages the temperature of the IV fluid through the arrangement between the fluid transfer line 3 and the plurality of heating wires 6.

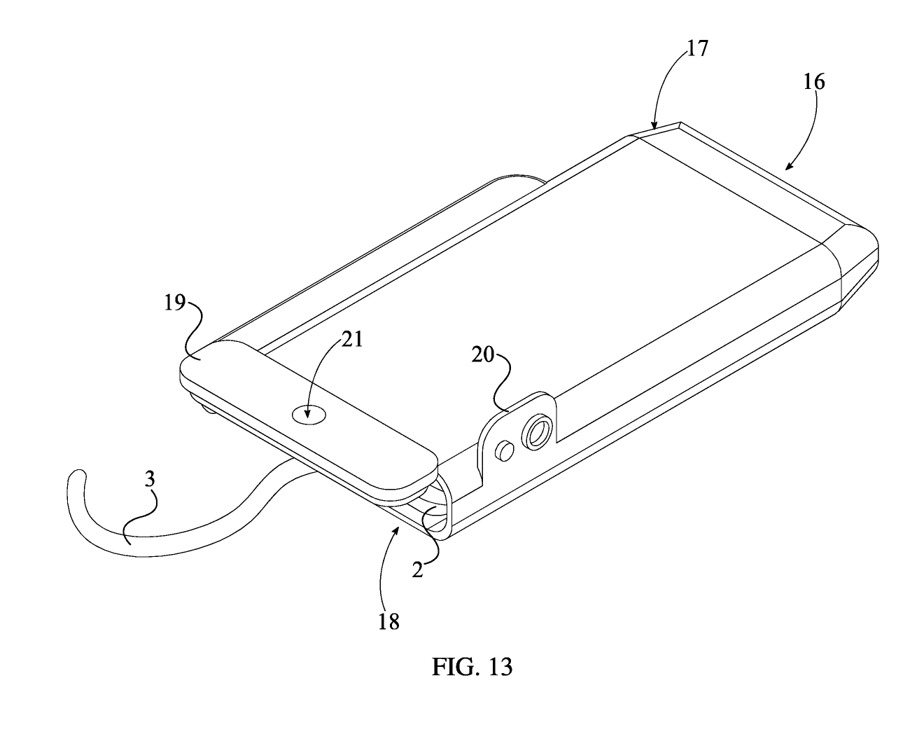

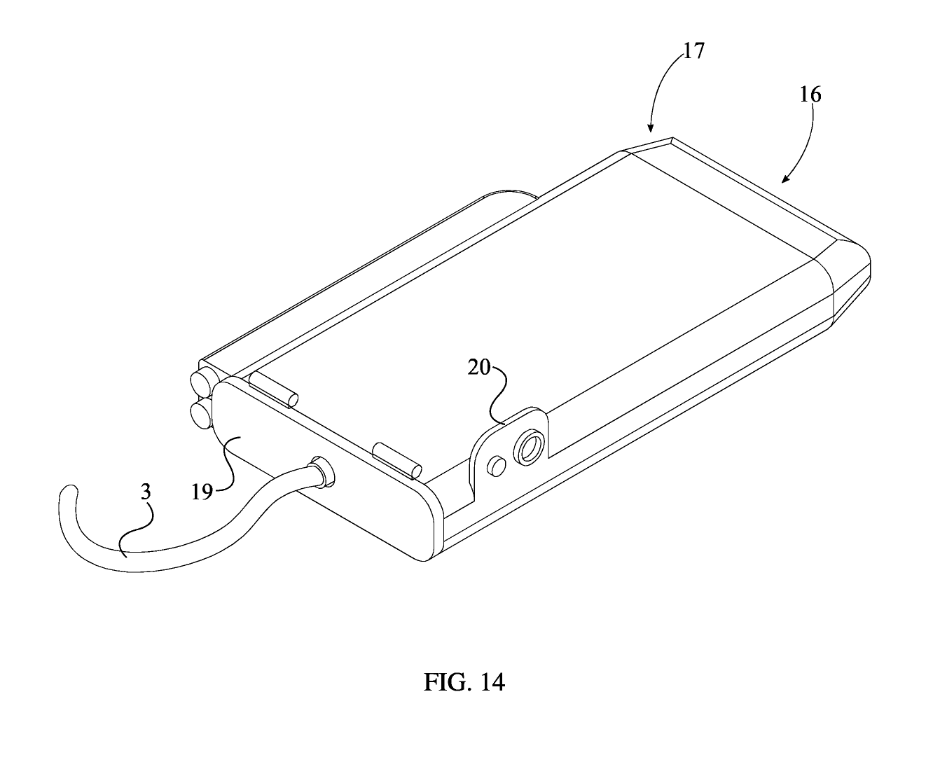

In order to control the temperature of the IV fluid throughout transport, a sleeve 16 houses the IV fluid retention unit 1 in an embodiment of the present invention. More specifically, the sleeve 16 houses the bag 2 of the IV fluid retention unit 1, as shown in FIG. 13. The sleeve 16 comprises an open end 17 and a closed end 18. The open end 17 allows the insertion of the bag 2 into the sleeve 16 and the closed end 18 prevents the bag 2 from escaping the sleeve 16. More specifically, the bag 2 is situated within the sleeve 16 through the open end 17. The microcontroller 4, the at least one heating mechanism 5, and the temperature sensor 7 is mounted within the sleeve 16. In a preferred embodiment of the present invention, a sealing mechanism 19 prevents the bag 2 within the sleeve 16 from escaping the sleeve 16, that is, the sealing mechanism 19 is operatively integrated into the sleeve 16, adjacent to the open end 17, wherein the sealing mechanism 19 is used to close off the open end 17. In the preferred embodiment of the present invention, the sealing mechanism 19 is a hinged cover with a lock. Various types of covers and locks may be in alternate embodiments of the present invention. Furthermore, a line-receiving hole 21 allows a bag 2 to be connected to a patient while contained within the sleeve 16 via a fluid transfer line 3. The line-receiving hole 21 laterally traverses into the sleeve 16, preferably through the sealing mechanism 19. The fluid transfer line 3 is positioned through the line-receiving hole 21, as shown in FIG. 14, and is in fluid communication with the bag 2 so that the fluid transfer line 3 delivers IV fluid to a patient from the bag 2 while positioned within the sleeve 16. This arrangement better maintains the temperature within the bag 2 as the bag 2 is within the sleeve 16. In another embodiment of the present invention, a pull tab 20 allows a user to maneuver the sleeve 16 and the bag 2 within the sleeve 16 throughout transport and while in storage. The pull tab 20 is externally fixed to the sleeve 16 facilitates the user's grasp around the sleeve 16.

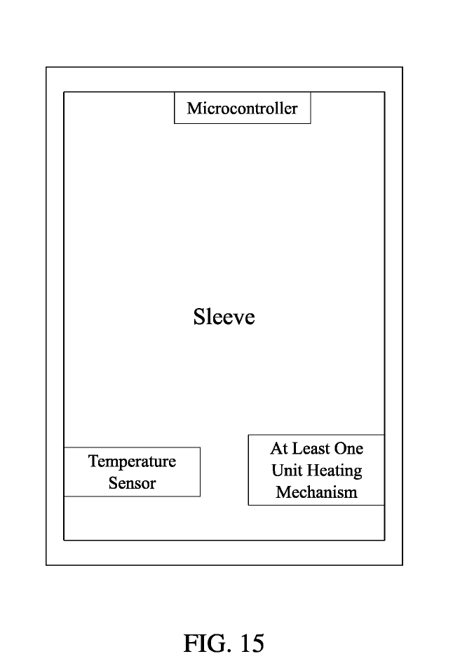

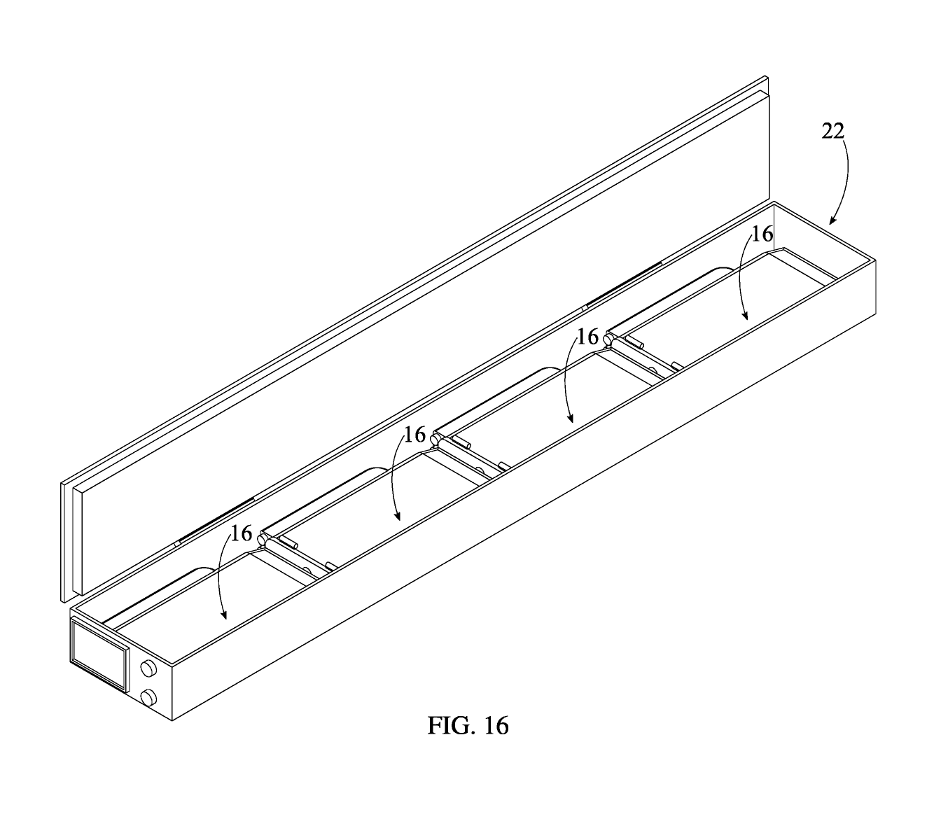

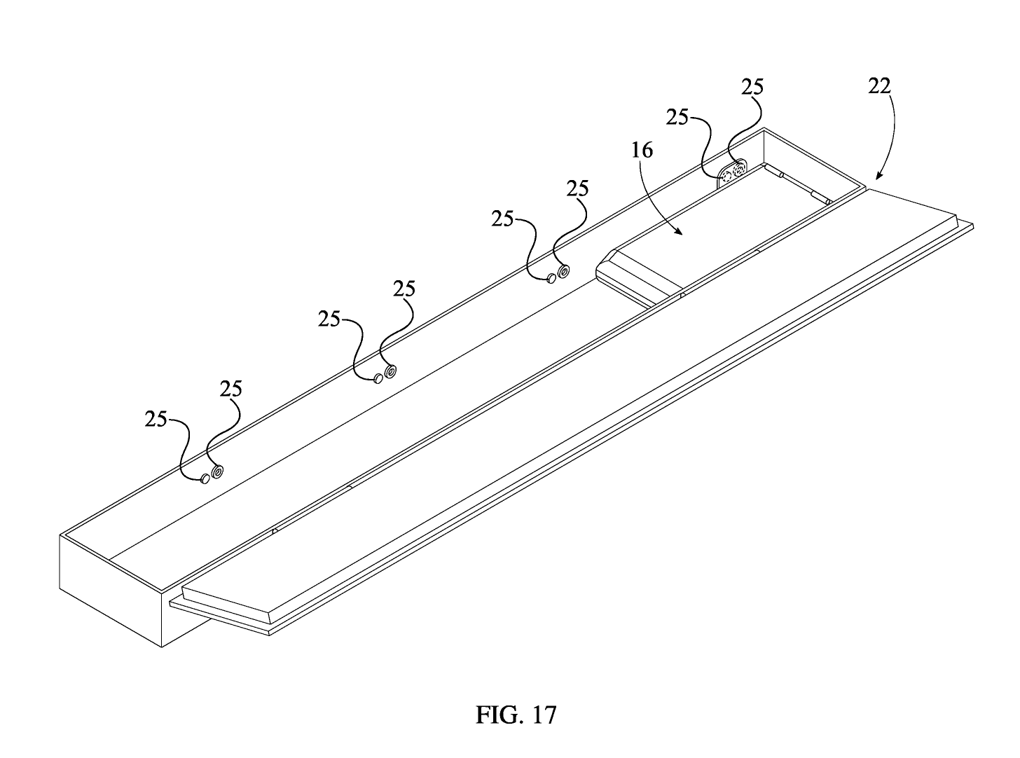

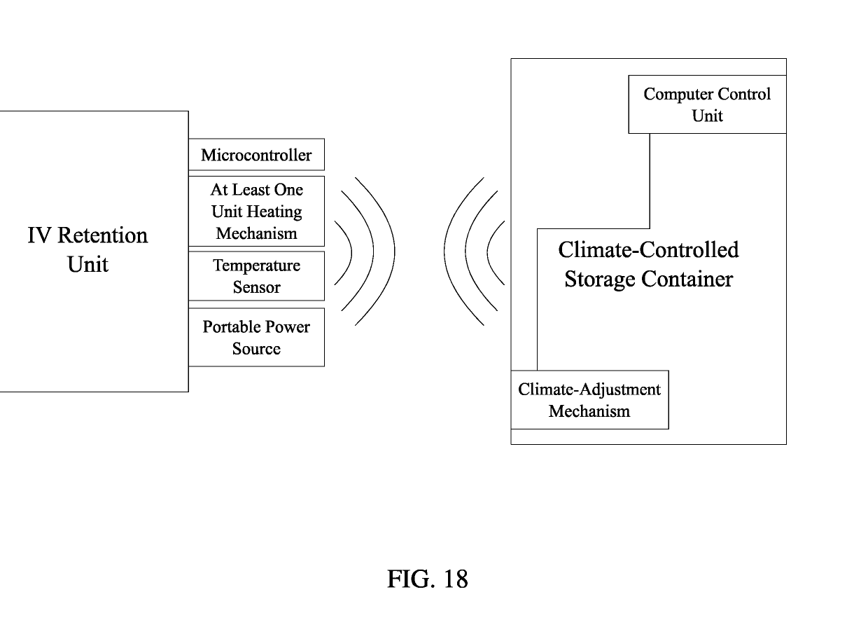

In order to control the temperature of the IV fluid while in storage, a climate-controlled storage container 22 houses and further insulates IV fluid. The IV fluid retention unit 1 is positioned within the climate-controlled storage container 22. The dimensions of the climate-controlled storage container 22 may vary in order to accommodate the stacking of a plurality of IV fluid retention units 1. In the preferred embodiment of the present invention, a plurality of power connectors 25 is mounted within the climate-controlled storage container 22, as shown in FIG. 17, thereby charging a plurality of IV fluid retention units 1. More specifically, the portable power source 8 is electrically coupled to a selected connector 25 from the plurality of power connectors 25. The selected connector not only charges the portable power source 8 but provides additional current and consequently heat which adjusts the temperature of the IV fluid, accordingly. In the preferred embodiment of the present invention, the portable power source 8 is a sleeve 16 which directly connects to a selected connector 25 via the pull tab 20. Furthermore, in the preferred embodiment of the present invention, the climate-controlled storage container 22 comprises a climate-adjustment mechanism 23. The climate-adjustment mechanism 23 and computer control unit 24 allows a user to manually adjust the temperature of the climate-controlled storage container 22 and remotely control the temperature of the climate-controlled storage container 22. The climate-adjustment mechanism 23 is electronically connected to the computer control unit 24, and the computer control unit 24 is communicably coupled to the microcontroller, as shown in FIG. 18. In another embodiment of the present invention, a sleeve 16 may house a bag 2 of an IV fluid retention unit 1. More specifically, the bag 2 is situated within the sleeve 16 through the open end 17 of the sleeve 16. In addition to the bag 2, the microcontroller 4, that at least one heating mechanism 5, and the temperature sensor 7 are mounted within the sleeve 16, as shown in FIG. 15. The sleeve 16 is mounted within the climate-controlled storage container 22 by the selected connector 25 so that the temperature of the IV fluid within the bag 2 is better regulated, as shown in FIG. 17. A plurality of sleeves 16 may be housed and charged within the climate-controlled storage container 22 as shown in FIG. 16.

Although the invention has been explained in relation to its preferred embodiment, it is to be understood that many other possible modifications and variations can be made without departing from the spirit and scope of the invention as hereinafter claimed.

What is claimed is:

1. A system of intravenous fluid temperature control comprises:
an intravenous (IV) fluid retention unit;
a microcontroller;
at least one heating mechanism;
a temperature sensor;
a portable power source;
the at least one heating mechanism and the temperature sensor being in thermal communication with the IV fluid retention unit;
the microcontroller being electronically connected to the at least one heating mechanism and the temperature sensor;
the microcontroller, the at least one heating mechanism, the temperature sensor, and the portable power source being externally mounted to the IV fluid retention unit; and
the portable power source being electrically connected to the microcontroller, the at least one heating mechanism, and the temperature sensor.

2. The system of intravenous fluid temperature control as claimed as claim 1 comprises:
a user input/output console;
the user input/output console being externally mounted onto the IV fluid retention unit; and
the user input/output console being electronically connected to the microcontroller.

3. The system of intravenous fluid temperature control as claimed as claim 1 comprises:
an inseparable housing;
the IV fluid retention unit comprises a bag;
the inseparable housing being externally connected onto the bag; and
the microcontroller, the at least one heating mechanism, the temperature sensor, the portable power source being mounted within the inseparable housing.

4. The system of intravenous fluid temperature control as claimed as claim 1 comprises:
a separable housing;
a disengageable fastener;
the IV fluid retention unit comprises a bag;
the separable housing being externally attached onto the bag by the disengageable fastener; and
the microcontroller, the at least one heating mechanism, the temperature sensor, and the portable power source being mounted within the separable housing.

5. The system of intravenous fluid temperature control as claimed as claim 1 comprises:
the IV fluid retention unit comprises a bag and a fluid transfer line;
the at least one heating mechanism comprises a plurality of heating wires;
the fluid transferring line being in fluid communication with the bag;
the plurality of heating wires being positioned along the fluid transfer line;
the plurality of heating wires being distributed around the fluid transfer line; and
the plurality of heating wires being laterally mounted to the fluid transfer line.

6. The system of intravenous fluid temperature control as claimed as claim 5 comprises:
a peristaltic pump;
the microcontroller being electronically connected to the peristaltic pump; and
the fluid transfer line being in fluid communication with the bag through the peristaltic pump.

7. The system of intravenous fluid temperature control as claimed as claim 5 comprises:
a flowrate sensor;
the microcontroller being electronically connected to the flowrate sensor; and
the flowrate sensor being operatively integrated along the fluid transfer line, wherein the flowrate sensor is used to measure a fluid flowrate through the fluid transfer line.

8. The system of intravenous fluid temperature control as claimed as claim 5 comprises:
an insulative sheath; and
the fluid transfer line and the plurality of heating wires being enclosed within the insulative sheath.

9. The system of intravenous fluid temperature control as claimed as claim 1 comprises:
a sleeve;
the sleeve comprises an open end and a closed end;
the IV fluid retention unit comprises a bag;
the bag being situated within the sleeve through the open end; and
the microcontroller, the at least one heating mechanism, and the temperature sensor being mounted within the sleeve.

10. The system of intravenous fluid temperature control as claimed as claim 9 comprises:
a sealing mechanism; and
the sealing mechanism being operatively integrated into the sleeve, adjacent to the open end, wherein the sealing mechanism is used to close off the open end.

11. The system of intravenous fluid temperature control as claimed as claim 9 comprises:
a pull tab; and
the pull tab being externally fixed to the sleeve.

12. The system of intravenous fluid temperature control as claimed as claim 9 comprises:
a line-receiving hole;
the IV fluid retention unit comprises a bag and a fluid transfer line;
the line-receiving hole laterally traversing into the sleeve;
the fluid transferring line being positioned through the line-receiving hole; and
the fluid transferring line being in fluid communication with the bag.

13. The system of intravenous fluid temperature control as claimed as claim 1 comprises:
a climate-controlled storage container;
a plurality of power connectors;
the plurality of power connectors being mounted within the climate-controlled storage container;
the IV fluid retention unit being positioned within the climate-controlled storage container; and
the portable power source being electrically coupled to a selected connector from the plurality of power connectors.

14. The system of intravenous fluid temperature control as claimed as claim 13 comprises:
a sleeve;
the sleeve comprises an open end and a closed end;
the IV fluid retention unit comprises a bag;
the bag being situated within the sleeve through the open end;
the microcontroller, the at least one heating mechanism, and the temperature sensor being mounted within the sleeve; and
the sleeve being mounted within the climate-controlled storage container by the selected connector.

15. The system of intravenous fluid temperature control as claimed as claim 13 comprises:
the climate-controlled storage container comprises a climate-adjustment mechanism and a computer control unit;
the climate-adjustment mechanism being electronically connected to the computer control unit; and
the computer control unit being communicably coupled to the microcontroller.
